SEQUENCE LISTING (11 sequences)

SEQ ID NO: 1 - DNA, 4021 bp, Artificial Sequence
  plasmid
tcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccccgcggggatccatggaaggaaaaaagcggccgcaaaaggaaaactagtctagattaatacgactcactatagggagacccaagctggctagagcttgatatcgaattccccagatctgggggatcgatcctgagaacttcagggtgagtttggggacccttgattgttctttctttttcgctattgtaaaattcatgttatatggagggggcaaagttttcagggtgttgtttagaatgggaagatgtcccttgtatcaccatgcatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattaattcactcctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccgtcgcgggatctgcccgggcgtttaaacgccgcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcatggctgactaattttttttatttatgcagaggccgaggccgcctcggcctctgagctattccagaagtagtgaggaggcttttttggagg

SEQ ID NO: 2 - DNA, 6811 bp, Artificial Sequence
  plasmid
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggcccctctgcaggataattcggcacgaggctgtgttagaggtgaaccatcttaattactagttctattacctaattcagcttccttgtttggtctgctgtggatctgccttattgcatatgccatgcatcagataatggatgcatcagataatggtgttagacaaagcttcattgtgaacaacctaatgcattttagagaaacaatctcatcacattttttctagcctttcctacatttaaacttgctgttgcccaaattataattttttaaatgtctttggtgggcttctgttaattcacatgacttgagcttatagctatgtctactgcacagattgggtaatggaacactaaacttttatacttgaaaatgacagccttaaatgctcatatcagtcacaaatctaggatgtactgtcttgttgtatgtgagctttgtagagatttttaaaaatataagcatcaccttcccattgaagagtggagagagtctactggatgactggccaggaactttctctctgaatcggacatttggatgtcttctttcttccaagaaatggtggttcacattaaagtatcatggccttatgtatgctcaaatggaatcttatgtaactttcttatttaattttggtctgcttatttttagataaaattgaaaggaattgtataaatcaattaacatattagctgagttgtccaacacatggtataaacgaattacaacagtaaactattacacatttccaaaaaaaaaaaaaaaaaagcggccgcaagcttggtacctctagaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgatcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcctcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 3 - DNA, 93 bp, Artificial Sequence
  plasmid fragment
aaaaggcctatataagcagagctcgtttagtgaaccgtcagatcgcctggagacgccatccacgctgttttgacctccccgcggggatcccct

SEQ ID NO: 4 - DNA, 93 bp, Artificial Sequence
  plasmid fragment
ttttccggatatattcgtctcgagcaaatcacttggcagtctagcggacctctgcggtaggtgcgacaaaactggaggggcgcccctagggga

SEQ ID NO: 5 - DNA, 93 bp, Artificial Sequence
  plasmid fragment
cgcggatccatggaaggaaaaaagcggccgcaaaaggaaaactagtctagattaatacgactcactatagggagacccaagctggctagctag

SEQ ID NO: 6 - DNA, 93 bp, Artificial Sequence
  plasmid fragment
gcgcctaggtaccttccttttttcgccggcgttttccttttgatcagatctaattatgctgagtgatatccctctgggttcgaccgatcgatc

SEQ ID NO: 7 - DNA, 56 bp, Artificial Sequence
  plasmid fragment
tacaggcctctgcaggatatcctcgaggcggccgcaagcttggtacctctagagca

SEQ ID NO: 8 - DNA, 56 bp, Artificial Sequence
  plasmid fragment
atgtccggagacgtcctataggagctccgccggcgttcgaaccatggagatctcgt

SEQ ID NO: 9 - DNA, 18 bp, Artificial Sequence
  antisense oligonucleotide
tgcattaggttgttcaca

SEQ ID NO: 10 - DNA, 18 bp, Artificial Sequence
  antisense oligonucleotide
tgcagtagtttttgcaca

SEQ ID NO: 11 - DNA, 18 bp, Artificial Sequence
  antisense oligonucleotide
ccttacctgctagctggc

(19) United States
(12) Patent Application Publication
(10) Pub. No.: US 2003/0124523 A1
(43) Pub. Date: Jul. 3, 2003

(54) Organic compounds

(76) Inventors: Fredericus Alphonsus Maria Asselbergs, Riehen (CH); Jonathan Hall, Reinach (CH); Dieter Huesken, Freiburg i. Br. (DE); Bernd Kinzel, Loerrach (DE); Francois Natt, Aesch (CH); Jan Weiler, Loerrach (DE)

Correspondence Address: Thomas Hoxie, Novartis Corporation, Patent and Trademark Dept., 564 Morris Avenue, Summit, NJ, 07901-1027, US

(21) Appl. No.: 09/883,573

(22) Filed: Jun. 18, 2001

Related U.S. Application Data
(60) Provisional application No. 60/213,132, filed on Jun. 22, 2000.
(60) Provisional application No. 60/266,949, filed on Feb. 7, 2001.

(51) Int. Cl. C12Q 1/68; C12Q 1/66; C12N 15/87

(52) U.S. Cl. 435/6; 435/455; 435/8; 435/320.1

(57) ABSTRACT

The invention relates to a reporter construct useful for the identification of oligo- or polynucleotides that modulate the expression of a target nucleic acid. In particular, in one embodiment, it is directed to a screening assay for the identification of oligo- or polynucleotides that modulate the expression of a target nucleic acid.

The invention relates to a reporter construct useful for the identification of oligo- or polynucleotides that modulate the expression of a target nucleic acid. In particular, in one embodiment, it is directed to a screening assay for the identification of oligo- or polynucleotides that modulate the expression of a target nucleic acid.

The search for new drug targets in the pharmaceutical industry within the functional genomics arena requires a high throughput approach to allow large numbers of genes to be assessed for their suitability as new drug targets (Dyer et al., Drug Discovery Today 1999, 4(3), 109-114). The use of antisense oligonucleotides as tools in functional assays is a potent method of assessment. Antisense oligonucleotides directed at a given mRNA target, whether the target is an mRNA from a well characterised cDNA or simply an EST sequence representing a novel gene of which little else is known, downregulate expression of the gene and provide an opportunity to study the biological consequences of the inhibition. The antisense approach to drug target identification involves three main steps:

First, selection of target genes to be assessed as suitable new pharmaceutical targets;

Second, identification of biologically-active antisense oligonucleotides capable of lowering levels of expression of the said target genes;

Third, testing said antisense oligonucleotides in a functional assay to determine the biological consequences of reducing expression levels of the said gene.

A slow step in this process is the second step.

There are several possible mechanisms of action of antisense oligonucleotides (De Mesmaeker et al, Acc. Chem. Res. 1995, 28(9), 366-74), of which the principle one is that of induced mRNA cleavage by RNase H. In brief, the antisense oligonucleotide binds to the mRNA target thus creating a hybrid duplex which is recognised by the ubiquitous cellular enzyme RNase H. Induction of RNase H leads to a rapid and apparently irreversible cleavage of the mRNA strand, thus resulting in a reduction of the mRNA level in the cell. Only antisense oligonucleotides with a particular kind of chemical constitution are capable of inducing RNase H, in particular those antisense oligonucleotides containing stretches of phosphorothioated DNA are of special interest because of their wide applicability. There are also numerous reports in the literature of antisense oligonucleotides which are biologically active through a mechanism which does not entail cleavage of the mRNA (Baker et al., J. Biol. Chem. 1997, 272(18), 11994-12000), for example steric blocking of the mRNA translation process, particularly those targetting the AUG regions or in the 5′-UTR (untranslated region). This activity can not easily be detected by studying levels of the target mRNA in the cell during or after the antisense oligonucleotide treatment as in many cases it remains unchanged by the antisense oligonucleotide treatment. In fact, the biological activity of such an antisense oligonucleotide is most usually only detected at the protein level: the protein level is decreased while the mRNA level remains unchanged.

It is presently not possible to predict a priori whether an antisense oligonucleotide will operate by the RNase H cleavage type mechanism, or whether a steric blocking will be effected, even though the chemical composition of the antisense oligonucleotide may be capable of inducing cleavage of its target mRNA through the RNase H mechanism. However, in a study of the mechanism of a series of active antisense oligonucleotides it was found that those antisense oligonucleotides targetting the 3′-UTR of a mRNA do so by activating RNase H, thus causing a detectable reduction of the mRNA level (Crooke, Stanley T. Medical Intelligence Unit: Therapeutic Applications of Oligonucleotides 1995, 138 pp, page 44).

It is inadvisable to try and draw conclusions concerning any phenotypic changes observed in an antisense experiment without checking that the antisense oligonucleotide has in fact lowered levels of the target mRNA/protein: there are numerous reports of antisense oligonucleotides causing non-specific effects in cellular assays (Stein C. A. , Antisense and Nucleic Acid Drug Development 1998, 8(2), 129-32).

A first step in the analysis of a gene as a new drug target using antisense technology is the selection of a suitable biologically active antisense oligonucleotide. If an mRNA for example has a length of approximately 5000 nucleotides (nts), and a typical active antisense oligonucleotide of 20 nt is selected, then there are approximately 5000 possible different antisense oligonucleotides available. Most of the antisense oligonucleotides complementary to a given mRNA target are, for a number of possible reasons, biologically inactive. A biologically active antisense oligonucleotide has to be shown experimentally. The potency of an antisense oligonucleotide during the selection process is determined by studying the levels of the gene expression at the mRNA level or at the protein level after the antisense oligonucleotide treatment. Although, ultimately, it is the effects of the protein downregulation which determine the biological consequences of an antisense treatment, measuring mRNA levels is considerably easier experimentally, especially in a rapid throughput approach. Furthermore, the assumption that protein levels decrease relative to mRNA levels is usually borne out. Measurement of target protein levels require antibodies, relatively large numbers of treated cells, and also knowledge of the protein sequence. Measurement of mRNA levels, on the other hand, can be performed with techniques more amenable to rapid throughput and consequently, remains the method of choice for determining which from a series of antisense oligonucleotide sequences are in fact biologically active in assays. Active antisense oligonucleotides which function by mechanisms other than RNase H cleavage (also decay) are in the rapid throughput setting less useful because detection of antisense activity requires target protein level determination, and all of the disadvantages mentioned above associated with it.

Algorithms exist to predict antisense oligonucleotides which should show biological activity through a predicted accessible binding site on the target mRNA (Walton et al., Biotechnology and Bioengineering 1999, 65(1), 1-9). To date however, the programmes are not sufficiently accurate to predict one antisense oligonucleotide sequence with “guaranteed” activity. Furthermore, even if this were successful, the algorithm has only predicted binding activity and not biological activity. Experimental techniques to determine binding activity exist, but these are for the main part laborious to perform, and also do not determine biological activity (Milner et al., Nat. Biotechnol. 1997, 15(6), 537-541). Experimental activities to determine antisense oligonucleotide sequences with biological activity from the use of combinatorial libraries of antisense oligonucleotides have been reported but as described above, are also too laborious to be workable in a high throughput setting (Ho et al., Nucleic Acids Res. 1992, 20(15), 3945-53). The surest way to identify biologically active antisense oligonucleotides is to test as many as possible in an antisense cell assay, monitoring levels of the target mRNA after a certain timepoint. A standard method of measuring mRNA levels is the northern blot and is labour intensive. A newer method is that of real time RT-PCR: this requires an expensive dedicated machine for measurement of fluorescence levels, and for each target mRNA a pair of DNA primer probes and an expensive TAQMAN probe (Sybr green, only primers). The RT-PCR reaction exploits the 5′-nuclease activity of AmpliTaq Gold DNA Polymerase to cleave a TAQMAN probe during PCR. The TAQMAN probe contains a reporter dye at the 5′-end of the probe and a quencher dye at the 3′-end of the probe. During the reaction, cleavage of the probe separates the reporter dye and the quencher dye resulting in increased fluorescence of the reporter dye. Accumulation of the PCR products is detected directly by monitoring the increase in in fluorescense of the reporter dye. For the testing of large numbers of antisense oligonucleotides extensive pipetting steps are required. For the testing of large numbers of antisense oligonucleotides against large numbers of different targets, extensive pipetting steps and multiple probes are required.

Some reporter assays for screening antisense oligonucleotides have been described (Vickers et al., Nucleic Acids Res. 1992, 20(15), 3945-53; Monia et al., Journal of Biological Chemistry 1992, 267(28), 19954-62; Caselmann et al., Intervirology 1998, 40(5-6), 394-399; U.S. Pat. No. 5,955,589; PCT Application No. WO 99/27135; PCT Application No. WO 94/08003). In all of these above, a luciferase reporter is fused 3′- to a target cDNA, or part of a target cDNA, whereby translation of the fusion results in a fusion protein comprising the reporter and the target. Inhibition of mRNA expression by an antisense mechanism is monitored indirectly by monitoring luciferase activity. In one example as an alternative reporter to luciferase beta-glucuronidase was used as a reporter inserted 3′-to the target cDNA of interest (C. Levis et al., Fr. Virus Genes 1992, 6(1), 33-46) for the screening of six antisense oligonucleotides for potency. For the investigation of single cDNA targets, this general type of assay represents a suitable method of determining the most potent antisense oligonucleotide from a series of oligonucleotides against a given target.

Only two examples of a target nucleic acid inserted 3′- to a reporter are known: In Poole et al. (Virology 1995, 206(1), 750-754), a target cDNA was inserted between two reporter genes in order to study features of cap-site dependent mRNA translation and cap-site independent RNA translation. In Vickers et al. (Nucleic Acids Res. 2000, 28,1340-1347), a synthetic target nucleic acid prepared by automated DNA synthesis is inserted 3′-to the luciferase reporter for analysing the structural features of the synthetic insert using one oligonucleotide.

For the effective use of antisense technology in the functional genomics setting, where success is heavily dependent on being able to apply rapid throughput techniques, there is no fast, reliable, cheap method of determining which from a large number of possible antisense oligonucleotides against a given target is the most potent and therefore most suitable as an antisense tool. Therefore, the method of measuring antisense oligonucleotide activity from a series of antisense oligonucleotides to determine the most potent compound assumes a key role in the throughput of antisense assays.

Although there are several methods to identify antisense oligonucleotides with biological activity, none of these is applicable in a high thoughput mode.

It is therefore desirable to provide an improved generally-applicable method which allows to a) efficiently analyse the biological activity of a series of multiple antisense oligonucleotides against given targets, b) monitor levels of mRNAs without the cost and the extensive pipetting associated with real time RT-PCR and c) avoid most, if not all, of the complications described above.

### SUMMARY OF THE INVENTION

The present invention relates to a reporter construct comprising a reporter element and a target nucleic acid inserted 3′- to the reporter element into the untranslated region.

Furthermore, the present invention relates to a process for the production of a reporter construct comprising a reporter element and a target nucleic acid wherein the target nucleic acid is inserted 3′- to the reporter element into the untranslated region.

The present invention also relates to the use of a reporter construct comprising a reporter element and a target nucleic acid inserted 3′- to the reporter element into the untranslated region in a method for the identification of biologically active oligo- or polynucleotides that modulate the expression of a target nucleic acid.

In another aspect the invention relates to screening assay for the identification of biologically active oligo- or polynucleotides that modulate the expression of a target nucleic acid comprising transfecting a reporter construct comprising a reporter element and a target nucleic acid inserted 3′- to the reporter element into the untranslated region and a candidate oligo- or polynucleotide into a suitable cell line and comparing the level of expression of the reporter protein when the reporter construct is transfected alone with the level of expression when the reporter construct and the oligo- or polynucleotide are transfected.

In a further aspect the invention relates to cells transfected with a reporter construct comprising a reporter element and a target nucleic acid inserted 3′- to the reporter element into the untranslated region.

### BRIEF DESCRIPTION OF THE DRAWINGS

FIG. 1 and FIG. 2 show plasmid maps of basic vector pNAS-016 and reporter vector pNAS-020. The firefly luciferase gene is inserted in the basic vector. Abbreviations: T7prom, bacterial T7 promoter; St-Xh, f, StuI-XhoI (fill in, Klenow-blunted) ligation site; Nh-Hi f, NheI-HindIII (fill in, Klenow-blunted) ligation site; SPLD-BG, splicing donor site of rabbit βglobin; SPLA-GB, splicing acceptor site of rabbit βglobin; pA-BG, polyadenylation site of rabbit βglobin; pBRori, origin of replication of pBR322; SV40ori, SV40 origin of replication. FIG. 3 shows the DNA sequence of pNAS-016. FIG. 4 shows the DNA sequence of pNAS-094.

### DETAILED DESCRIPTION OF THE INVENTION

The present invention relates to a reporter construct comprising a reporter element and a target nucleic acid inserted 3′- to the reporter element into the untranslated region. The reporter construct is nucleic acid based and the reporter element is functionally linked to the target nucleic acid such that binding of an oligo- or polynucleotide to target nucleic acid modulates the function or production of the reporter. Such modulation may be an increase or decrease of function or production. The level of function or production of the reporter is a direct measure for the effect of the binding of the oligo- or polynucleotide. The reporter element may have for instance a specific structure by itself that serves a specific function which is detectable such as e.g. interaction with a protein. The reporter element may also be for instance a nucleic acid molecule or a functional fragment thereof that encodes a protein or polypeptide that is capable of providing a detectable signal either on its own upon transcription or translation or by reaction with another one or more reagents. The reporter may e.g. code for an enzyme whose activity on its substrate is measurable in an assay. The reporter protein when expressed is detectable by means of a suitable assay procedure, e.g., by biological activity assay, enzyme-linked immunosorbent assay (ELISA) or radioimmunoassay (RIA). The nucleic acid molecule may be isolated from genomic DNA, such as a gene which may or may not contain introns, or a complementary DNA (cDNA) prepared using messenger RNA as a template. Reporter genes suitable for use herein are conventional in the art, selection of which is within the capability of a person skilled in the art. Examples of such reporter genes include that encoding the enzyme chloramphenicol acetyltransferase (CAT), the luc gene from the firefly that encodes luciferase, the bacterial lacZ gene from Escherichia coli that encodes P-galactosidase, alkaline phosphatase (AP), human growth hormone (hGH), the bacterial ss-glucuronidase (GUS), and green fluorescent protein (GFP). Preferred nucleic acid molecules are sequences that encode a light emitting reporter protein, preferrably a protein that is fluorescent. Preferred DNA sequences that encode a light emitting reporter protein code for GFP and light emitting derivatives thereof. GFP is from the jelly fish Aquorea victoria and is able to absorb blue light and re-emits an easily detectable green light and is thus suitable as a reporter protein. GFP may be advantageously used as a reporter protein because its measurement is simple and reagent free and the protein is non-toxic.

A reporter assay useful for the screening of antisense oligonucleotides requires the preparation of a reporter construct containing the target gene, or part of a target gene e.g. an EST. Such a vector can be constructed in different ways. For example, it is possible to make a vector: A. expressing a fusion protein where the target nucleic acid is inserted either in-frame, 5′- to the reporter, i.e. at the N-terminus, between START site and reporter coding region, or in-frame before the AUG start codon with its own new START site (Vickers et al., Nucleic Acids Res. 1992, 20(15), 3945-53; Monia et al., Journal of Biological Chemistry 1992, 267(28), 19954-62; Caselmann et al., Intervirology 1998, 40(5-6), 394-399; U.S. Pat. No. 5,955,589; PCT Application No. WO 99/27135; PCT Application No. WO 94/08003). B. expressing a fusion protein where the target nucleic acid is inserted in-frame 3′- to the reporter i.e. at the C-terminus, between reporter coding region and STOP signal; C. where the target nucleic acid is inserted out-of-frame lacking its own START site 5′- to the reporter with its START site, so that only the pure reporter protein is expressed (Le Tinévez et al., Nucleic Acids Res. 1998, 26(10), 2273-8; Vickers et al., Nucleic Acids Res. 2000, 28, 1340-1347).

In contrast to the above constructs the present invention relates to a reporter construct comprising a reporter element and a target nucleic acid inserted 3′- to the reporter element into the untranslated region. In such a construct the target nucleic acid is inserted independent of frame and STOP signals, 3′- to the reporter, after the STOP signal so that only the pure reporter protein is expressed.

For vectors of types A-C, care is needed with cloning as only specific regions of the target cDNA are suitable for use in the construct. This is not the case with the reporter construct of the present invention which offers significant advantages in terms of flexibility over the other examples.

For example in case of type A an insert from the 3′-UTR of a target cDNA, typically an EST, would not allow a fusion protein expression because of the numerous STOP signals that are inherent to 3′-UTRs. Alternatively, where an insert from the coding region of the target cDNA is selected, care would be needed to ensure that the reporter sequence be in-frame. In case of type B an insert from the 5′-UTR of a target cDNA would lead to a fusion protein with unfolded random-coil non-sense sequence at the C-terminus, resulting in degradation, toxicity, incorrect folding or other associated problems.

In case of type C an insert comprising the 5′-UTR with an AUG would give 1) where the AUG of the target insert is in frame with the reporter, a fusion protein of type A and 2) where the AUG of the target insert is out of frame with the reporter, no reporter peptide. In the case of the reporter construct of the present invention, whatever the origin of the target insert (5′UTR, AUG, coding, STOP, 3′UTR, intron) no special cloning requirements are required to ensure that translation leads to a fuctional reporter protein free of the aforementioned problems: the translated product of the vector is invariant, i.e. a pure reporter protein.

There are additional advantages over reporter constructs of type A and B. For example it is not necessary when proceeding from one target gene to another target gene and using the vector in a transient expression-type experiment to optimise for reporter expression. This remains approximately constant over all targets, simply because the expressed protein, i.e. the pure reporter, does not vary from target gene to target gene. In types A and B however, a unique fusion protein is generated for each new target cDNA used, leading to variations in expression levels, cellular localisations, half-lives, toxicities, etc. In addition, it is conceivable that the behaviour of both, the reporter and the protein of interest is unpredictably modified by the fusion. Consequently, each new fusion protein construct has to be validated as a biologically relevant model. This causes delays while experiments are conducted to optimise the fusion protein such that a satisfactory set of assay conditions are found for the antisense oligonucleotide screening process. These issues never arise with a reporter construct according to the present invention.

Consequently, such a vector represents a genuinely general type of reporter construct useful for the study of biological activity of antisense oligonucleotides or other oligo- or polynucleotides (e.g. ribozymes) which cause the decay of a target mRNA.

The target nucleic acid of the reporter construct can be any nucleic acid including DNA, RNA, cDNA, full length genes, full length cDNAs, and parts or fragments thereof such as DNA fragments or expressed sequence tags. The target nucleic acid may be of natural or synthetic origin, i.e. it may be e.g. isolated from cells or synthesized by an automated method known in the art. In a preferred embodiment of the present invention the target nucleic acid comprised in the reporter construct is a gene, a cDNA, a DNA fragment or an expressed sequence tag.

Reporter genes useful in the present invention allow the rapid and easy sreening of the effects of tested oligo- or polynucleotides on the expression of the target nucleic acid. The reporter gene may e.g. code for a cell surface protein that is easy to detect with e.g. an antibody directed to it. In another possibility the reporter may be an enhancer of a repressor protein such as e.g. the tetracyclin operon repressor protein. For example the repressor protein binds to the operon and kept another gene expression silent. After reduction of such an repressor construct a positive signal with less background can be measured as activity). Further useful examples are reporter genes coding for chloramphenicol acetyltransferase, alkaline phosphatase or beta-Galactose. In a preferred embodiment of the present invention the reporter gene codes for a fluorescent protein (e.g. fluorescent green, yellow, cyan, red, enhanced green, enhanced yellow, enhanced cyan, enhanced red). In another preferred embodiment of the present invention the reporter gene codes for yellow fluorescent protein, enhanced yellow fluorescent protein or luciferase.

In a further aspect the present invention relates to a process for the production of the reporter construct wherein a target nucleic acid is inserted 3′- to the reporter element into the untranslated region. The methods used for the production of the construct are well known to a person skilled in the art such as cloning technologies and can be obtained from standard textbooks or standard laboratory manuals such as for example Maniatis et al., Cold Spring Harbor Laboratory, Cold Spring Harbor, N.Y. 1989.

In another aspect the present invention relates to the use of the reporter construct in a method for the identification of biologically active oligo- or polynucleotides that modulate the expression of a target nucleic acid. Such a method may be for example a screening assay as described herein.

Accordingly, in a further aspect the present invention relates to a screening assay for the identification of biologically active oligo- or polynucleotides that modulate the expression of a target nucleic acid comprising transfecting the reporter construct and a candidate oligo- or polynucleotide into a suitable cell line and comparing the level of expression of the reporter protein when the reporter construct is transfected alone with the level of expression when the reporter construct and the oligo- or polynucleotide are transfected.

In another aspect the invention relates to cells transfected or transformed with a reporter construct comprising a reporter element and a target nucleic acid inserted 3′- to the reporter element into the untranslated region. A large number of eukaryontic cells of animal (e.g. Chinese Hamster Ovary cells) or human origin exist that are suitable for transfection with nucleic acids. Also encompassed by the present invention are prokayontic cells transformed with the reporter construct (e.g. bacterial cells such as E. coli). Suitable cells that can be used in the present invention are known to a person skilled in the art.

This screening assay allows to determine which from a series of oligo- or polynucleotides is the most biologically potent in terms of reducing the mRNA levels of a target nucleic acid, and therefore is the most suitable as a tool for an antisense method either as a tool for drug discovery, or a potential antisense oligonucleotide therapeutic. The assay is particularly well-suited to use in a rapid throughput to high throughput mode as:

1. Assays can be run in micro-titer well format;

2. Pipetting steps are kept to a minimum;

3. Readout may be done with light measurement directly from the 96-well format when for example a fluorescent reporter is used;

4. Readout is exactly the same for all targets. Each target does not require a unique set of expensive reagents such as TAQMAN probes, Sybr Green probes etc.

The present invention is particularly useful in cases where the complexity of a functional assays renders laborious the screening for an active oligo- or polynucleotide, e.g. using primary cells, or cells which are difficult to obtain, where the target mRNA is expressed endogenously at a very low level, or even where an in vitro assay does not exist and it is desired to use an oligo- or polynucleotide directly in an in vivo experiment. In such cases, the screening and identification of active oligo- or polynucleotides would be laborious or expensive in terms of material. The screening assay according to the present invention circumvents these problems.

The entire content of the references, patents and publications cited in this application is hereby incorporated by reference.

The invention is further described, for the purposes of illustration only, in the following examples.

### EXAMPLE 1

### Cloning

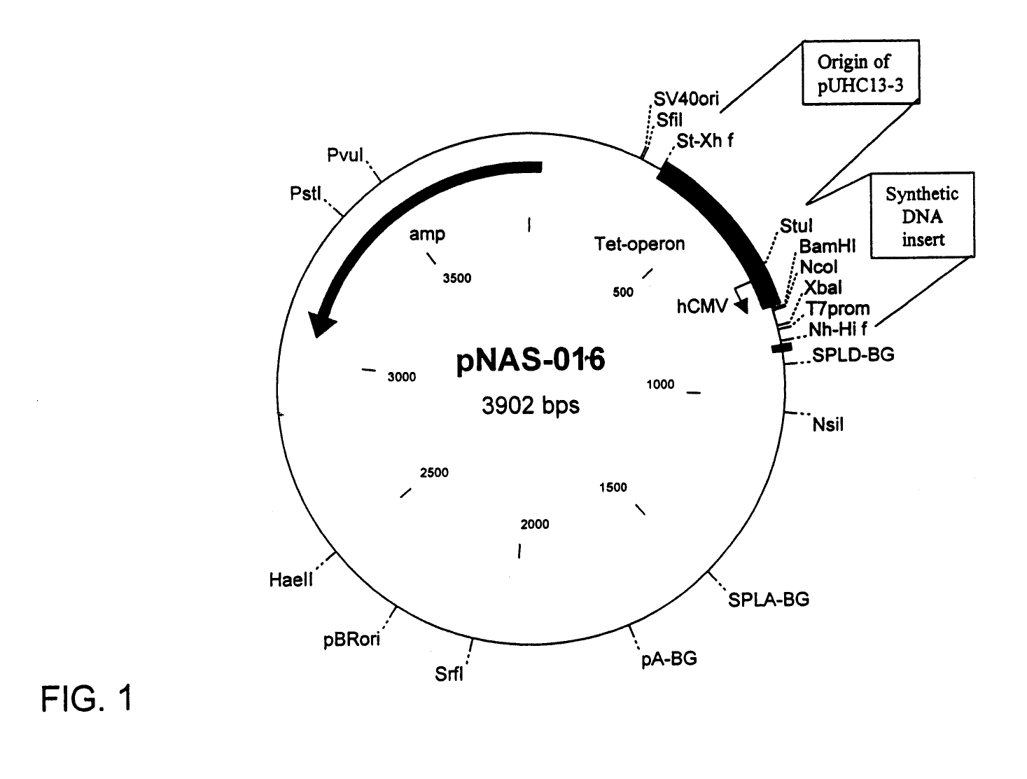

All plasmid manipulations are carried out according to standard methods (Maniatis et al., Cold Spring Harbor Laboratory, Cold Spring Harbor, N.Y. 1989). Expression vector pNAS-016 (FIG. 1) is constructed for inducible overexpression of reporter proteins and reporter-cDNA fused mRNAs of cloned cDNAs or ESTs as well as for in vitro run-off transcription of the cDNA. The origin of the vector is a plasmid (pSFhCMVT7neo1) which contains an SfiI restriction site cassette with the neo (geneticin-resistant) selection marker (also replaceable with other selection marker cassettes e.g. hpt (hygromycin phosphotransferase) and gpt (an E. coli enzyme, xanthine-guanine phosphoribosytransferase); cells can be selectively grown with xanthine in the presence of inhibitors aminopterin or mycophenolic acid) (Mulligan et al., Proc. Natl. Acad. Sci. U. S. A. 1981, 78(4), 2072-2076). After removing the neo cassette for easier further vector construction, the tetracycline operon (7 times repeated) and a part of the human minimal CMV promoter sequence (origin of plasmid pUHC13-3) (Magalini et al., DNA Cell Biol. 1995, 14(8), 665-761.) is replaced between the two StuI sites.

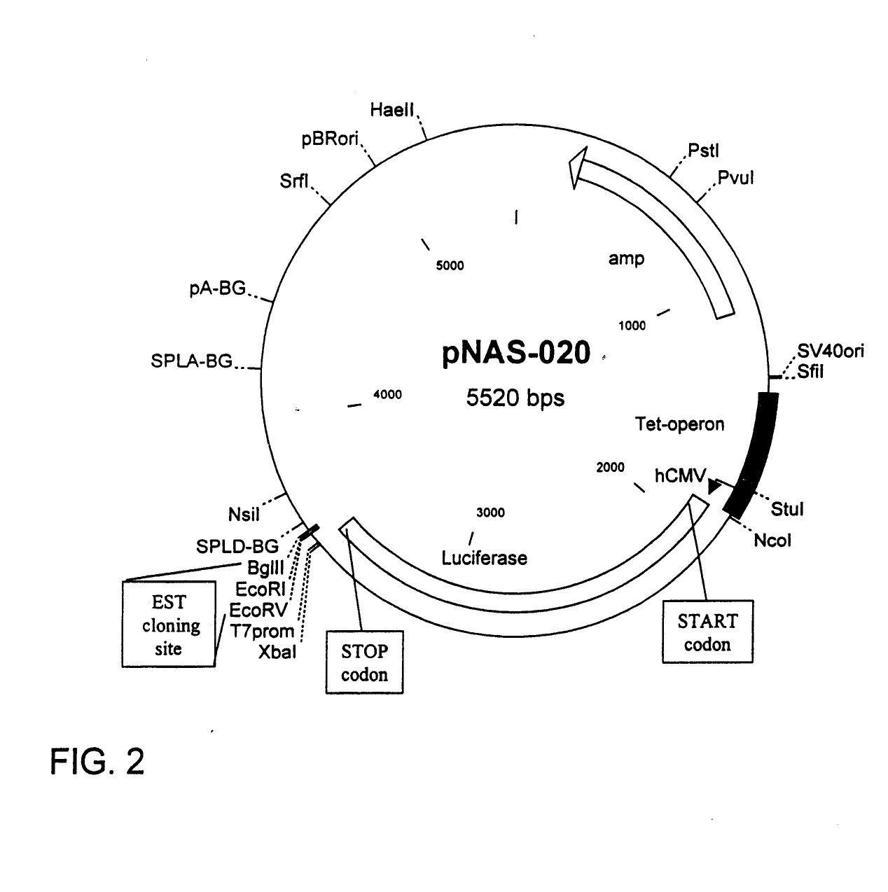

In addition, a synthetic DNA part is placed between the StuI and Hind III site. The synthetic DNA contains the transcription start of the eukaryotic mRNA and the bacterial T7 promoter to allow generating in vitro run-off transcripts. After inserting the firefly luciferase gene (pGL3 control vector, Promega) at the NcoI/XbaI site the vector pNAS-20 (FIG. 2) is obtained. For the antisense oligonucleotide screening the individual EST sequence is inserted at the EST cloning site (BgIII, EcoRI, EcoRV).

The plasmid pSFhCMVT7neo1 is digested with SfiI and religated to remove the neo resistance gene resulting in pNAS-003. For construction of clone pNAS-016, the SacI/XhoI fragment (301bp) of pUHC13-3 containing the tet operon is filled in at the XhoI site and ligated with the large fragment (3613 bp) of the plasmid pNAS-003 (StuI/SacI) to obtain pNAS-005. The small SacI fragment (53 bp), also from plasmid pUHC13-3, is ligated at the Sac I site of pNAS-005 resulting in pNAS-006. The right orientation of the insert in pNAS-006 is given by a restriction enzyme cut of SFII and KpnI, resulting of a 353 bp fragment. pNAS-006 is cut with StuI and HindIII and prior to ligation the Hind III site in the plasmid fragment (3857bp) is destroyed by filling in the ends with Klenow polymerase. Four synthetic DNA sequences are hybridized to two double stranded DNA fragments (5′AAAAGGCCTATATMGCAGAGCTCGTTTAGTGAACCGTCAGATCGCCTGGAGACGCCAT CCACGCTGTTTTGACCTCCCCGCGGGGATCCCCT3′; (SEQ. ID NO. 3) complementary:

| | |
|---|---|
| 3′TTTTCCGGATATATTCGTCTCGAGCAAATCACTTGGCAGTCTAGCGGACCTCTGCGGTAG | (SEQ. ID NO.4) |
| GTGCGACAAAACTGGAGGGGCGCCCCTAGGGGA5′; and | |
| 5′CGCGGATCCATGGAAGGAAAAAAGCGGCCGCAAAAGGAAAACTAGTCTAGATTAATACGA | (SEQ. ID NO.5) |
| CTCACTATAGGGAGACCCAAGCTGGCTAGCTAG3′; | |

complementary: 3′GCGCCTAGGTACCTTCCTIIIIlCGCCGGCGTTTTCCTTTTGATCAGATCTAATTATGCTG AGTGATATCCCTCTGGGTTCGACCGATCGATC5′) (SEQ. ID NO. 6) and each are treated with BamHI, ligated and treated with StuI and NheI resulting in a 160 bp fragment. After filling in the ends of NheI with Klenow polymerase the synthetic DNA fragment is blunt-end ligated into the prepared pNAS-006. The right orientation is given with a still cleaveable StuI site of pNAS-016. Clone pNAS-020 is obtained by ligation of the firefly luciferase gene into pNAS-016 at the NcoI and XbaI site. DNA clones used for the final reporter assay are constructed by inserting into the EST cloning site the c-DNA fragment of the EST clone respectively.

### EXAMPLE 2

### Cell Lines and Culture

Genetic background: SSF-3 cell is a CHO (chinese hamster ovary) cell line, derived from the dihydrofolate reductase (dhfr)-minus CHO line DUKXB11, which has acquired the ability to grow in a basal medium completely devoid of proteins (Gandor et al., FEBS Lett. 1995, 377(3), 290-294). A recombinant line of SSF-3 bearing the tetracycline responsive transactivator protein (tTA) and the mutant hamster dihydrofolate reductase as selection marker (methotrexate resistance) is used. tTA is compatible with the reporter vector pNAS-020 for constitutive luciferase expression. SSF-3 cells are grown as adherent cells in Cho-master medium HEPES buffered (Messi Cell Culture Technology, Zürich, Switzerland ,#CG-051) containing 10% bovine calf serum (BCS) (Life Technol., #16170-086) in 5% humidified CO₂ atmosphere at 37° C. Alternatively SSF-3 cells can be cultured in suspension in the synthetic Cho-master medium without serum. Stable cells expressing the red shifted green fluorescent protein (pd2EGFP-N1, Clontech; lipofectamine-PLUS, #10964-013 transfection according to the manufacture, Life Technologies Inc.) are selected as neo+clones by addition of 1 mg/ml geneticin.

H1299 cells (ATCC collection (CRL-5803)) are neuroendocrine non-small cell lung carcinoma cells, which express the autocrine growth factor neuromedin B. The cells are grown in RPMI 1640 medium (Life Technologies #21875-034) supplemented with 10% BCS (Life Technol., #16170-086) in a 5% humidified CO2 atmosphere at 37° C.

### EXAMPLE 3

### Transfection of Expression Plasmids and Oligonucleotides

Lipofectamine-PLUS (lipofectamine-PLUS, Life Technologies #10964-013)/plasmid mixture: Plasmids are prepared by the QIAfilter plasmid maxi kit (Qiagen, #12262) and stored at 1 μg/ml in TE (10 mM Tris pH 8.0, 1 mM EDTA). Lipofectamine is diluted in OptiMEM-I (Life Technol. #31985-039) 25 fold (40 μl/ml). A second solution of OptiMEM-I is prepared containing the plasmid and the PLUS reagent. The plasmid is diluted 50 fold (20 ng/μl) and the PLUS reagent is diluted 16.7 fold (60 μl/ml). Both solutions are left at room temperature for 15 min. A 1:1 mixture of the two solutions is prepared and left for 15 min. The mixture is 5-fold diluted with OptiMEM-I to 2-fold of the final concentration (1 ng/μl for the plasmid; 2 μl/ml lipofectamine) before usage in the well. The final concentration of the lipofection reagent is 5.6 μM lipofectamine (bilipid equivalents).

Lipofectin (lipofectin, Life Technol. #18292-011)/oligonucleotide mixture: Oligonucleotides are stored at 1 mM concentration in water and pre-diluted to 400 μM in 0.2 mM HEPES (4-(2-hydroxyethyl)-piperazine-1-ethane-sulfonic acid) buffer at pH 6.5. All oligonucleotides are diluted in OptiMEM-I 40 fold (10 μM). Separately lipofectin (1 mg/ml, 1:1 mixture (w/w) of DOPE & DOTMA) is diluted 2.5 fold in OptiMEM-I (400 μl/ml); both solutions are left at room temperature for 30 min. A 1:1 mixture of the two solutions is prepared and left for 10 min. The mixture is 4.17-fold further diluted with OptiMEM-I to 3-fold of the final concentration (400 nM for the oligonucleotides; 4 μl/ml lipofectin/100 nM oligonucleotide) before adding to the well. The final concentration of the lipofection reagent can be deduced as: 23 μM lipofectin (bilipid equivalents) or 11 μM cationic lipid (DOTMA) or positive charge equivalents. The final concentration of the oligonucleotides can be deduced as: 400 nM oligonucleotide or 0.165 μM negative charge equivalents. The ratio of positive charge equivalents to negative charge equivalents is 68:1 and of bilipid equivalents to oligonucleotide equivalents 58:1.

For the luciferase reporter assays, cells are split 48 h hours prior to transfection reaching approx. 1.5×10⁷ SSF-3 cells/150 cm² flask. Cells are treated with trypsin-EDTA (Life Technologies #25300-054), suspended in Cho-master medium (HEPES buffered; Dr. Messi Cell Culture Technology ,#CG-051) containing 10% bovine calf serum (BCS) (Life Technologies, #16170-086), counted, centrifuged and suspended in OptiMEM-I at 35000 cells/50 μl . For the transfection the lipofectamine-PLUS-plasmid mixture and the cell suspension are combined (50 μl from each) and plated in Costar 96-well assay plates (white, clear bottom, #3610) and incubated for 2 hours in 5% humidified CO₂ atmosphere at 37° C. 50 μl of the prepared lipofectin-antisense oligonucleotide mixture is then added to the cell monolayer which is then incubated for 2 h in the CO₂ incubator. The medium is removed and replaced with 100 μl standard Cho-master medium containing 10% BCS and incubated over night. The green fluorescent protein expression, from living cells is measured at each manipulation step to confirm adherence.

For real time PCR assays one day prior to the transfection 2×10⁵ H1299 cells/well are plated in 6 well assay plates. Oligonucleotides are stored at 100 μM concentration in TE (10 mM Tris pH 8.0, 1 mM EDTA). All oligonucleotides are diluted in OptiMEM-I 125-fold (0.8 μM). Separately, lipofectin (1 mg/ml, 1:1 mixture (w/w) of DOPE & DOTMA) is diluted 83.3-fold in OptiMEM-I (12 μl/ml) and left at room temperature for 30 min. A 1:1 mixture with the final concentration (400 nM for the oligonucleotides; 1.5 μl/mi lipofectin/100 nM oligonucleotide) is prepared and left for 15 min. before adding to the cells after medium had been aspirated. The final concentration of the lipofection reagent can be deduced as: 8.6 μM lipofectin (bilipid equivalents) or 4.1 μM cationic lipid (DOTMA) or positive charge equivalents. The final concentration of the oligonucleotides can be deduced as: 400 nM oligonucleotide or 0.165 μM negative charge equivalents. The ratio of positive charge equivalents to negative charge equivalents is 25:1 and of bilipid equivalents to oligonucleotide equivalents 22:1. Cells are transfected for 4 h in a final volume of 1 ml. After transfection the culture medium is aspirated, 3 ml RPMI 1640 medium containing 10% bovine calf serum is added, and the cells are incubated in 5% humidified C0₂ atmosphere at 37° C. for 20 h.

### EXAMPLE 4

### Antisense Oligonucleotides

All antisense oligonucleotides are selected as 18-mer hemi-mer formats, for example: CsAsTsTsAsTsTsGsCscscstsgsasasasg, with the following abbreviations: s=phosphorothioate linkage; small lettering=2′-O-methoxy-ethyl oligoribonucleotide modified. The sequences are listed in Table 2. From each target number the corresponding EST clone identifier number is included in the file name (Table 1).

TABLE 1
Target nucleic acids

| Target no. | (ATTC) EST clone identifier |
|---|---|
| #4 | CloneID: 310021 Origin: human fibroblasts, senescent |
| #5 | CloneID: 487407 Origin: human uterus (pregnant), adult |
| #7 | CloneID: 487909 Origin: human uterus (pregnant), adult |
| #8 | CloneID: 276699 Origin: human lesions (4), |
| | one male, 46 years |
| #16 | CloneID: 487433 Origin: human uterus (pregnant), adult |
| #32 | CloneID: 486086 Origin: human uterus (pregnant), adult |

TABLE 2
Antisense oligonucleotides

| NAS | Target | CloneID | Sequence |
|---|---|---|---|
| 5048.1 | #4 | CloneID310021 | TsCsCs TsGsTs GsCsGs |
| | | | tststs cscsgs tsasg |
| 5049.1 | #4 | CloneID310021 | TsGsTs TsCsCs TsGsTs |
| | | | gscsgs tststs cscsg |
| 5050.1 | #4 | CloneID310021 | AsAsCs TsCsCs CsAsCs |
| | | | cstsgs cscsas cstsg |
| 5051.1 | #4 | CloneID310021 | CsTsCs CsAsTs GsCsTs |
| | | | gsgscs ascsts tsgsa |
| 5052.1 | #4 | CloneID310021 | GsCsCs TsCsCs AsCsCs |
| | | | tstsgs tstsgs asast |
| 5053.1 | #4 | CloneID310021 | TsCsTs CsTsCs CsAsTs |
| | | | gstscs cstscs asasa |
| 5054.1 | #4 | CloneID310021 | GsCsAs TsCsTs GsTsCs |
| | | | csgscs tsgsgs gscsg |
| 5055.1 | #4 | CloneID310021 | CsTsCs AsCsCs GsGsCs |
| | | | csasts csascs tstsg |
| 5056.1 | #4 | CloneID310021 | GsCsTs CsTsCs CsGsCs |
| | | | asgscs tscsas cscsg |
| 5057.1 | #4 | CloneID310021 | TsCsCs CsAsCs TsCsGs |
| | | | cscsts tscscs astsg |
| 5343.1 | #5 | CloneID487407 | GsAsGs AsAsCs CsTsTs |
| | | | cstscs tscsgs asasc |
| 5344.1 | #5 | CloneID487407 | TsCsCs TsCsCs AsGsGs |
| | | | csasgs csascs tsgsa |
| 5345.1 | #5 | CloneID487407 | GsCsTs CsAsCs AsGsGs |
| | | | csasas gststs cscst |
| 5346.1 | #5 | CloneID487407 | TsCsCs AsAsGs AsCsAs |
| | | | tststs cscscs tscsa |
| 5347.1 | #5 | CloneID487407 | TsAsAs CsTsCs CsAsGs |
| | | | gsasas cststs asasa |
| 5348.1 | #5 | CloneID487407 | TsGsCs TsGsAs CsAsTs |
| | | | cststs csasts tsgsg |
| 5349.1 | #5 | CloneID487407 | CsGsCs TsGsCs TsTsTs |
| | | | csasts cstsas astsa |
| 5350.1 | #5 | CloneID487407 | TsTsCs AsCsTs CsGsCs |
| | | | tsgscs tststs csast |
| 5351.1 | #5 | CloneID487407 | TsGsCs GsTsGs AsTsCs |
| | | | asasgs tscsts gstst |
| 5352.1 | #5 | CloneID487407 | TsGsTs GsTsGs CsGsTs |
| | | | gsasts csasas gstsc |
| 5094.1 | #7 | CloneID487909 | AsAsGs TsTsAs TsCsCs |
| | | | csascs csasts tstsa |
| 5095.1 | #7 | CloneID487909 | TsCsTs CsAsTs GsGsTs |
| | | | csasas csasas ascst |
| 5096.1 | #7 | CloneID487909 | TsCsTs CsTsCs AsCsAs |
| | | | asasts gstscs gscst |
| 5097.1 | #7 | CloneID487909 | TsCsCs CsTsTs GsAsAs |
| | | | cscsts gscsts cstsg |
| 5098.1 | #7 | CloneID487909 | AsAsCs CsAsCs AsCsAs |
| | | | astscs asascs tscsa |
| 5099.1 | #7 | CloneID487909 | AsCsAs GsCsAs CsAsGs |
| | | | ascscs cscsas cscsa |
| 5100.1 | #7 | CloneID487909 | CsGsCs TsGsCs TsCsAs |
| | | | cscsas tscscs tsgsc |
| 5101.1 | #7 | CloneID487909 | CsCsCs TsAsCs AsAsTs |
| | | | aststs tscscs tsgsa |
| 5102.1 | #7 | CloneID487909 | TsCsTs CsCsCs TsAsCs |
| | | | asasts aststs tscsc |
| 5103.1 | #7 | CloneID487909 | TsCsCs AsTsAs AsTsCs |
| | | | tscsas tscsts astst |
| 5058.1 | #8 | CloneID276699 | CsCsTs TsCsCs TsCsTs |
| | | | tsgsts gscsts csasa |
| 5059.1 | #8 | CloneID276699 | CsAsCs CsCsTs GsGsTs |
| | | | ascsas gstscs csgsc |
| 5060.1 | #8 | CloneID276699 | CsAsCs CsGsGs CsAsCs |
| | | | cscsts gsgsts ascsa |
| 5061.1 | #8 | CloneID276699 | AsCsCs CsTsCs CsCsTs |
| | | | tsgsgs gsascs cscst |
| 5062.1 | #8 | CloneID276699 | GsAsCs CsCsAs GsAsCs |
| | | | cscsts cscscs tstsg |
| 5063.1 | #8 | CloneID276699 | AsCsAs TsTsGs CsAsAs |
| | | | ascsas csasgs gsasa |
| 5064.1 | #8 | CloneID276699 | GsTsTs CsAsGs TsAsCs |
| | | | tstscs ascscs asasa |
| 5065.1 | #8 | CloneID276699 | TsAsCs AsCsAs CsCsTs |
| | | | gscsts cscsas gscst |
| 5066.1 | #8 | CloneID276699 | GsGsCs AsCsCs CsTsGs |
| | | | gstsas csasgs tscsc |
| 5067.1 | #8 | CloneID276699 | CsCsCs TsAsAs TsCsTs |
| | | | ascscs tscscs tscsa |
| 5108.1 | #16 | CloneID487433 | AsGsTs GsTsCs TsGsCs |
| | | | tscsts tscsas tsgsa |
| 5109.1 | #16 | CloneID487433 | AsCsCs AsAsCs GsCsCs |
| | | | tsgscs cscsts cscsc |
| 5110.1 | #16 | CloneID487433 | TsGsCs AsCsTs CsCsAs |
| | | | gsgscs gscscs asgsg |
| 5111.1 | #16 | CloneID487433 | CsCsTs TsAsGs TsGsTs |
| | | | cscsas csgsts gsast |
| 5112.1 | #16 | CloneID487433 | CsGsTs GsCsCs TsTsAs |
| | | | gstsgs tscscs ascsg |
| 5113.1 | #16 | CloneID487433 | GsAsCs GsGsAs TsGsGs |
| | | | ascsas tsasas tscsa |
| 5114.1 | #16 | CloneID487433 | GsGsCs TsAsGs TsGsTs |
| | | | gscsas tstsas tstst |
| 5115.1 | #16 | CloneID487433 | GsGsTs TsGsTs CsAsGs |
| | | | asgsgs cstsas gstsg |
| 5116.1 | #16 | CloneID487433 | AsAsGs TsTsCs AsGsAs |
| | | | cscscs ascsas tsgst |
| 5117.1 | #16 | CloneID487433 | TsAsCs TsGsTs GsAsCs |
| | | | csgsas gstscs tsasc |
| 5558 | #32 | CloneID486086 | tgc atTs AsGsGs TsTsGs |
| | | | Tstc aca |
| 5734 | #32 | CloneID486086 | tgc agTs AsGsTs TsTsTs |
| | | | Tsgc aca |
| 5596 | #32 | CloneID486086 | cct taCs CsTsGs CsTsAs |
| | | | Gsct ggc |

### EXAMPLE 5

### Firefly Luciferase/Green Fluorescent Protein (eGFP) Assay

All parameters are measured on the multifunctional microtiter plate reader Victor-2™ (Wallac). The green fluorescent protein expression from living cells is measured at several time points to follow the growth and at the end point after 22 hours (not including the 4 h transfection period) for the cell number unit. For the end point measurement the assay plate is centrifuged for 8 min at 1500 rpm and the culture medium is aspirated. The plate is placed into the Victor-2™ and the fluorescence is measured with the emission filter of 485 nm±15 nm and the excitation filter of 510 nm±10 nm.

The luciferase activity is measured by lysing the cells in 50 μl passive lysis buffer (Promega, #E1941) and incubated by gently shaking for 1 h at room temperature. The plate (COSTAR, white, clear bottom #3610) is placed into the Victor-2™ and 100 μl luciferase substrate reagent per well (Promega #E148A) is injected immediately before light measurement. The instrument is set on ‘injection flash mode’ with a delay time of 1 sec (after substrate injection) and an integration time of 10 sec. The output value is in RLU (relative light units). With a calibration of the expressing GFP SSF-3 cells the GFP fluorescence can be converted to cell number or used as the denominator in the quotient of luminometer units (RLU, luciferase) per fluorimeter units (GFP). The quotient expresses the luciferase activity per cell. Read-out was after 24 h. Results are presented as % of luciferase mismatch control sequence (4535, CsCsTs TsAsCs CsTsGs cstsas gscsts gsgsc)±13.6% (Table 3).

TABLE 3
Antisense oligonucleotides activity in the reporter assay of example 5

| NAS | Target | % of control | NAS | Target | % of control | NAS | Target | % of control |
|---|---|---|---|---|---|---|---|---|
| 5048.1 | #4 | 51.0 | 5350.1 | #5 | 81.5 | 5062.1 | #8 | 109.3 |
| 5049.1 | #4 | 48.0 | 5351.1 | #5 | 60.4 | 5063.1 | #8 | 81.2 |
| 5050.1 | #4 | 45.8 | 5352.1 | #5 | 57.8 | 5064.1 | #8 | 67.9 |
| 5051.1 | #4 | 34.7 | 5094.1 | #7 | 71.6 | 5065.1 | #8 | 62.4 |
| 5052.1 | #4 | 27.7 | 5095.1 | #7 | 55.0 | 5066.1 | #8 | 47.3 |
| 5053.1 | #4 | 44.8 | 5096.1 | #7 | 51.8 | 5067.1 | #8 | 60.4 |
| 5054.1 | #4 | 55.3 | 5097.1 | #7 | 52.4 | 5108.1 | #16 | 45.7 |
| 5055.1 | #4 | 38.3 | 5098.1 | #7 | 64.2 | 5109.1 | #16 | 61.3 |
| 5056.1 | #4 | 48.2 | 5099.1 | #7 | 82.5 | 5110.1 | #16 | 95.1 |
| 5057.1 | #4 | 39.5 | 5100.1 | #7 | 75.8 | 5111.1 | #16 | 44.7 |
| 5343.1 | #5 | 59.3 | 5101.1 | #7 | 95.1 | 5112.1 | #16 | 65.3 |
| 5344.1 | #5 | 45.1 | 5102.1 | #7 | 96.1 | 5113.1 | #16 | 65.7 |
| 5345.1 | #5 | 52.1 | 5103.1 | #7 | 102.2 | 5114.1 | #16 | 67.0 |
| 5346.1 | #5 | 61.6 | 5058.1 | #8 | 83.6 | 5115.1 | #16 | 79.5 |
| 5347.1 | #5 | 72.8 | 5059.1 | #8 | 63.9 | 5116.1 | #16 | 60.9 |
| 5348.1 | #5 | 71.7 | 5060.1 | #8 | 80.3 | 5117.1 | #16 | 62.1 |
| 5349.1 | #5 | 55.5 | 5061.1 | #8 | 73.3 | | | |

### EXAMPLE 6

### Real Time PCR/Total RNA Assay

Total RNA is extracted using the RNeasy 96 kit (Qiagen #74183). Primer pairs and FAM-labelled TAQMAN probes for real time PCR are designed using the Primer Express v1.0 program (ABI PRISM, PE Biosystems) and purchased from Birsner & Grob (primers) or Perkin Elmer (TAQMAN probes). For the real time PCR reaction 50 ng total RNA is mixed with 5′ and 3′ primers (10 μM each), TAQMAN probe (5 μM), MuLV reverse transcriptase (6.25 u, PE Biosystems), RNase Out RNase inhibitor (10 u, Life Technologies #10777-019) and the components of the TAQMAN PCR reagent kit (PE Biosystems #N808-0228) in a total volume of 25 μl following the TAQMAN PCR reagent kit protocol (PE Biosystems). Reverse transcription and real time PCR is performed in a ABI PRISM sequence detector 7700 (PE Biosystems) as follows: 2 minutes reverse transcription at 50° C., 10 minutes denaturation at 95° C. followed by 50 cycles of denaturation for 15 sec. at 95° C. and annealing and elongation for 1 min at 60° C. The relative quantitation of gene expression is calculated as described in the ABI PRISM 7700 user bulletin #2 (PE Biosystems).

### EXAMPLE 7

### Green Fluorescent Expressing SSF-3 Cell Line

Stable cell lines of the SSF-3 line (tTA+, dhfr+) are generated with expression of the green fluorescent protein under the human CMV promoter by geneticin (neo) selection. The purpose of using GFP expressing cells is to establish a practical measurement of the cell number. On one hand it is possible to monitor each physical manipulation of the cells during the different adding and replacing steps of liquid in the assay, and on the other hand, the GFP measurement serves for the normalisation of the luciferase activity value per cell.

By testing different microtiter plates especially for adherent cell culture purpose (Costar plates) or for suspension cell culture purpose (Millipore plates with transparent filter bottom) a linear correlation between the fluorescence unit and the cell number is observed. In both plates the values is linear up to 1.2×10⁶ seeded cells per well. However, these results are only obtained by using the bottom read option with the scan mode of the fluorometer Victor-2™. This scan mode allows one to measure each part of the whole well bottom taking into account the heterogeneous distribution of the cells on the well bottom. In the scan mode nine data points are generated with a beam of an area size of 3 mm in diameter.

### EXAMPLE 8

### Lipofection

Reproducible day to day results and a considerable amount of reduction of the luciferase expression after 22 hours is achieved using lipofectamine and the PLUS reagent for the plasmid transfection and adding the lipofectin oligonucleotide transfection mixture after 2 hours. Again after 2 hours all reagents are replaced with medium containing 10% BCS.

### EXAMPLE 9

### Green Fluorescent Protein and Luciferase Read-out

Relative activities are measured in triplicates of independent experiments from each antisense oligonucleotide complementary to an EST. Each of the oligonucleotides are also tested against a non-related target. The values are the ratio of luciferase unit per GFP unit in relation to the mismatch control against the luciferase reporter as 100%. The luciferase RLU (relative light units) are normalised with the green fluorescent protein fluorescence unit. Read-out is 22 hours after transfection and reproduced in an independent experiment after one week. The quality of each run is controlled by two positive controls (an antisense oligonucleotide complementary to the luciferase coding region and against the human CMV transcription start) and two negative controls (a three mismatch version of the luciferase matched oligonucleotides and a mixture of five non related antisense oligonucleotides), the cells untreated and the cells only treated with lipofectin. In addition, the day to day correlation plot indicates the high level of day to day reproducibility.

The assessment of the reporter assay as a reliable method for the measurement of the relative activity of an antisense oligonucleotide against its complementary RNA is done by comparison of the relative activity of the same antisense oligonucleotides in a reference assay. The reference assay is performed by the treatment of H1299 cells but in this case the target is the natural endogenous mRNA, and mRNA levels are counted by real time PCR and normalised against the total RNA amount. Five series of ten antisense oligonucleotides each targetting an EST are tested in both assays. A very good correlation is seen between the results of down-regulation of the pure reporter protein and that of the natural endogenous full length functional mRNA. From a set of antisense oligonucleotides, those antisense oligonucleotides which are observed to be the most active in the cellular reporter assay are also seen to be the most active on the endogenous mRNA, when assayed with real-time RT-PCR.

### EXAMPLE 10.1

### Cloning of pNAS-094

pNAS-094 contains within a single vector two reporter genes: the blue fluorescent protein for a normalisation measurement and yellow fluorescent protein to monitor antisense activity of antisense oligonucleotides to be tested. As transfection efficiency of oligonucleotides and plasmid DNA varies between individual cells, the use of a single vector ensures that this variable is eliminated in the experimental analysis thus adding accuracy to determination of oligonucleotide potency. Preparation of a standard transfectant (see Example 2) is not necessary when using this vector.

All plasmid manipulations are carried out according to standard methods (Maniatis et al., Cold Spring Harbor Laboratory, Cold Spring Harbor, N.Y. 1989). Expression vector pNAS-094 (FIG. 3) is constructed for overexpression of reporter proteins and reporter-cDNA fused mRNAs of cloned cDNAs or ESTs.

The origin of the vector is a plasmid (pBUDCE4, Invitrogen #V532-20) which contains a CMV and a EF-1 alpha promoter and the zeo selection marker.

After inserting the cyan-fluorescent protein gene (pECFP-N1, Clontech #6900-1) as a SmaI/NotI(fill-in) fragment at the NotI(fill-in)/XhoI(fill-in) site of pBUDCE4 the vector pNAS-90 is obtained. After inserting the yellow fluorescent protein gene (pEYFP-N1, Clontech #6006-1) as BamHI/NotI fragment at the BamHI/NotI site of pcDNA4/TO (Invitrogen #V1020-20) the vector pNAS-55 is obtained.

After inserting a multiple cloning site as a synthetic StuI/XbaI fragment at the NotI(fill-in)/XbaI site of pNAS-55 the vector pNAS-89 is obtained (synthetic complementary DNA sequences 5′TACAGGCCTCTGCAGGATATCCTCGAGGCGGCCGCAAGCTTGGTACCTCTAGAGCA3′ (SEQ. ID NO. 7) and: 3′ATGTCCGGAGACGTCCTATAGGAGCTCCGCCGGCGTTCGAACCATGGAGATCTCGT5′ (SEQ. ID NO. 8) are cut with StuI/XbaI). After inserting the yellow fluorescent protein gene from pNAS-89 as BamHI(fill-in)/XbaI fragment at the HindIII(fill-in)/XbaI site of pNAS-90 the vector pNAS-92 is obtained.

After inserting the EST (target insert #32) from the ATCC clone (ATCC 943180; CloneID: 486086; Origin: human uterus (pregnant), adult) as a EcoRI(fill-in)/NotI fragment at the EcoRV, NotI site of pNAS-92 the vector pNAS-094 is obtained.

### EXAMPLE 10.2

### Cell Lines and Culture

KB-3-1 (a human cervix carcinoma) line was used to demonstrate the effectiveness of the construct. KB-3-1 cells are grown as adherent cells in α-MEM (Life Technologies #32571-028) containing 5% fetal bovin serum (FBS) (Life Technologies, #16140-071) in 5% humidified CO₂ atmosphere at 37° C.

### EXAMPLE 10.3

### Transfection of Expression Plasmids and Oligonucleotides

Lipofectamine-PLUS (lipofectamine-PLUS, Life Technologies #10964-013)/plasmid mixture: Plasmids are prepared by the QIAfilter plasmid maxi kit (Qiagen, #12262) and stored at 1 μg/ml in TE (10 mM Tris pH 8.0, 1 mM EDTA). Lipofectamine is diluted in OptiMEM-I (Life Technol. #31985-039) 25 fold (40 μl/ml). A second solution of OptiMEM-I is prepared containing the plasmid and the PLUS reagent. The plasmid is diluted 50 fold (20 ng/μl) and the PLUS reagent is diluted 16.7 fold (60 μl/ml). Both solutions are left at room temperature for 15 min. A 1:1 mixture of the two solutions is prepared and left for 15 min. The mixture is 5-fold diluted with OptiMEM-I to 2-fold of the final concentration (1 ng/μl for the plasmid; 2 μl/ml lipofectamine) before usage in the well. The final concentration of the lipofection reagent is 5.6 μM lipofectamine (bilipid equivalents).

Lipofectin (lipofectin, Life Technol. #18292-011)/oligonucleotide mixture: Oligonucleotides are stored at 1 mM concentration in water and pre-diluted to 400 μM in 0.2 mM HEPES (4-(2-hydroxyethyl)-piperazine-1-ethane-sulfonic acid) buffer at pH 6.5. All oligonucleotides are diluted in OptiMEM-I 40 fold (10 μM). Separately lipofectin (1 mg/ml, 1:1 mixture (w/w) of DOPE & DOTMA) is diluted 2.5 fold in OptiMEM-I (400 μl/ml); both solutions are left at room temperature for 30 min. A 1:1 mixture of the two solutions is prepared and left for 10 min. The mixture is 4.17-fold further diluted with OptiMEM-I to 3-fold of the final concentration (400 nM for the oligonucleotides; 4 μl/ml lipofectin/100 nM oligonucleotide) before adding to the well. The final concentration of the lipofection reagent can be deduced as: 23 μM lipofectin (bilipid equivalents) or 11 μM cationic lipid (DOTMA) or positive charge equivalents. The final concentration of the oligonucleotides can be deduced as: 400 nM oligonucleotide or 0.165 μM negative charge equivalents. The ratio of positive charge equivalents to negative charge equivalents is 68:1 and of bilipid equivalents to oligonucleotide equivalents 58:1.

For the reporter assays, confluent cells in T-75 flask are split 24 h hours prior to transfection. Cells are treated with trypsin-EDTA (Life Technologies #25300-054), suspended in α-MEM (Life Technologies #32571-028) containing 5% fetal bovin serum (FBS) (Life Technologies, #16140-071), counted, centrifuged and suspended in OptiMEM-I at 30000 cells/50 μl. For the transfection the lipofectamine-PLUS-plasmid mixture and the cell suspension are combined (50 μl from each) and plated in Costar 96-well assay plates (black, clear bottom, #3603) and incubated for 2 hours in 5% humidified CO₂ atmosphere at 37° C. 50 μl, of the prepared lipofectin-antisense oligonucleotide mixture is then added to the cell monolayer which is then incubated for 2 h in the CO₂ incubator. The medium is removed and replaced with 100 μl standard α-MEM medium without phenolred (Life Technologies #41061-029) containing 5% FBS and incubated over night. The fluorescent protein expression (cyan and yellow) from living cells is measured at several time points with Ex filter 436±20 nm and Em filter 480±30 nm and Ex filter 500±25 and Em filter 535±30 respectively.

### EXAMPLE 10.4

### Antisense Assay of pNAS-094

The following oligonucleotides were used in an antisense assay:

| | | |
|---|---|---|
| 5558, antisense: | TGCATTAGGTTGTTCACA | (SEQ. ID NO.9) |
| 5734, mismatch: | TGCAGTAGTTTTTGCACA | (SEQ. ID NO.10) |
| 5596, control: | CCTTACCTGCTAGCTGGC | (SEQ. ID NO.11) |

Read-out was after 48 h. Results are presented as % of unrelated control sequence (5596):

5558: 65.32±12.75

5734: 121.30±14.46

5596: 100.00±8.78

FIG. 3: DNA sequence of pNAS-016
TCGAGTTTACCACTCCCTATCAGTGATAGAGAAAAGTGAAAGTCGAGTTT
ACCACTCCCTATCAGTGATAGAGAAAAGTGAAAGTCGAGTTTACCACTCC
CTATCAGTGATAGAGAAAAGTGAAAGTCGAGTTTACCACTCCCTATCAGT
GATAGAGAAAAGTGAAAGTCGAGTTTACCACTCCCTATCAGTGATAGAGA
AAAGTGAAAGTCGAGTTTACCACTCCCTATCAGTGATAGAGAAAAGTGAA
AGTCGAGTTTACCACTCCCTATCAGTGATAGAGAAAAGTGAAAGTCGAGC
TCGGTACCCGGGTCGAGTAGGCGTGTACGGTGGGAGGCCTATATAAGCAG
AGCTCGTTTAGTGAACCGTCAGATCGCCTGGAGACGCCATCCACGCTGTT
TTGACCTCCCCGCGGGGATCCATGGAAGGAAAAAAGCGGCCGCAAAAGGA
AAACTAGTCTAGATTAATACGACTCACTATAGGGAGACCCAAGCTGGCTA
GAGCTTGATATCGAATTCCCCAGATCTGGGGGATCGATCCTGAGAACTTC
AGGGTGAGTTTGGGGACCCTTGATTGTTCTTTCTTTTTCGCTATTGTAAA
ATTCATGTTATATGGAGGGGGCAAAGTTTTCAGGGTGTTGTTTAGAATGG
GAAGATGTCCCTTGTATCACCATGCATGGACCCTCATGATAATTTTGTTT
CTTTCACTTTCTACTCTGTTGACAACCATTGTCTCCTCTTATTTTCTTTT
CATTTTCTGTAACTTTTTCGTTAAACTTTAGCTTGCATTTGTAACGAATT
TTTAAATTCACTTTTGTTTATTTGTCAGATTGTAAGTACTTTCTCTAATC
ACTTTTTTTTCAAGGCAATCAGGGTATATTATATTGTACTTCAGCACAGT
TTTAGAGAACAATTGTTATAATTAAATGATAAGGTAGAATATTTCTGCAT
ATAAATTCTGGCTGGCGTGGAAATATTCTTATTGGTAGAAACAACTACAT
CCTGGTCATCATCCTGCCTTTCTCTTTATGGTTACAATGATATACACTGT
TTGAGATGAGGATAAAATACTCTGAGTCCAAACCGGGCCCCTCTGCTAAC
CATGTTCATGCCTTCTTCTTTTTCCTACAGCTCCTGGGCAACGTGCTGGT
TATTGTGCTGTCTCATCATTTTGGCAAAGAATTAATTCACTCCTCAGGTG
CAGGCTGCCTATCAGAAGGTGGTGGCTGGTGTGGCCAATGCCCTGGCTCA
CAAATACCACTGAGATCGATCTTTTTCCCTCTGCCAAAAATTATGGGGAC
ATCATGAAGCCCCTTGAGCATCTGACTTCTGGCTAATAAAGGAAATTTAT
TTTCATTGCAATAGTGTGTTGGAATTTTTTGTGTCTCTCACTCGGAAGGA
CATATGGGAGGGCAAATCATTTAAAACATCAGAATGAGTATTTGGTTTAG
AGTTTGGCAACATATGCCCATATGCTGGCTGCCATGAACAAAGGTTGGCT
ATAAAGAGGTCATCAGTATATGAAACAGCCCCCTGCTGTCCATTCCTTAT
TCCATAGAAAAGCCTTGACTTGAGGTTAGATTTTTTTTATATTTTGTTTT
GTGTTATTTTTTTCTTTAACATCCCTAAAATTTTCCTTACATGTTTTACT
AGCCAGATTTTTCCTCCTCTCCTGACTACTCCCAGTCATAGCTGTCCCTC
TTCTCTTATGGAGATCCGTCGCGGGATCTGCCCGGGCGTTTAAACGCCGC
GGCACCTCGCTAACGGATTCACCACTCCAAGAATTGGAGCCAATCAATTC
TTGCGGAGAACTGTGAATGCGCAAACCAACCCTTGGCAGAACATATCCAT
CGCGTCCGCCATCTCCAGCAGCCGCACGCGGCGCATCTCGGGGCCGACGC
GCTGGGCTACGTCTTGCTGGCGTTCGCGACGCGAGGCTGGATGGCCTTCC
CCATTATGATTCTTCTCGCTTCCGGCGGCATCGGGATGCCCGCGTTGCAG
GCCATGCTGTCCAGGCAGGTAGATGACGACCATCAGGGACAGCTTCAAGG
CCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAG
AGGTGGCGAAACCCGACAGGACTATAAAGATACCAGGCGTTTCCCCCTGG
AAGCTCCCTCGTGCGCTCTCCTGTTCCGACCCTGCCGCTTACCGGATACC
TGTCCGCCTTTCTCCCTTCGGGAAGCGTGGCGCTTTCTCAATGCTCACGC
TGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATC
GTCTTGAGTCCAACCCGGTAAGACACGACTTATCGCCACTGGCAGCAGCC
ACTGGTAACAGGATTAGCAGAGCGAGGTATGTAGGCGGTGCTACAGAGTT
CTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCT
TTTCTACGGGGTCTGACGCTCAGTGGAACGAAAACTCACGTTAAGGGATT
TTGGTCATGAGATTATCAAAAAGGATCTTCACCTAGATCCTTTTAAATTA
AAAATGAAGTTTTAAATCAATCTAAAGTATATATGAGTAAACTTGGTCTG
ACAGTTACCAATGCTTAATCAGTGAGGCACCTATCTCAGCGATCTGTCTA
TTTCGTTCATCCATAGTTGCCTGACTCCCCGTCGTGTAGATAACTACGAT
ACGGGAGGGCTTACCATCTGGCCCCAGTGCTGCAATGATACCGCGAGACC
CACGCTCACCGGCTCCAGATTTATCAGCAATAAACCAGCCAGCCGGAAGG
GCCGAGCGCAGAAGTGGTCCTGCAACTTTATCCGCCTCCATCCAGTCTAT
TAATTGTTGCCGGGAAGCTAGAGTAAGTAGTTCGCCAGTTAATAGTTTGC
GCAACGTTGTTGCCATTGCTGCAGGCATCGTGGTGTCACGCTCGTCGTTT
GGTATGGCTTCATTCAGCTCCGGTTCCCAACGATCAAGGCGAGTTACATG
ATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCA
CTGCATAATTCTCTTACTGTCATGCCATCCGTAAGATGCTTTTCTGTGAC
TGGTGAGTACTCAACCAAGTCATTCTGAGAATAGTGTATGCGGCGACCGA
GTTGCTCTTGCCCGGCGTCAACACGGGATAATACCGCGCCACATAGCAGA
ACTTTAAAAGTGCTCATCATTGGAAAACGTTCTTCGGGGCGAAAACTCTC
AAGGATCTTACCGCTGTTGAGATCCAGTTCGATGTAACCCACTCGTGCAC
CCAACTGATCTTCAGCATCTTTTACTTTCACCAGCGTTTCTGGGTGAGCA
AAAACAGGAAGGCAAAATGCCGCAAAAAAGGGAATAAGGGCGACACGGAA
ATGTTGAATACTCATACTCTTCCTTTTTCAATATTATTGAAGCATTTATC
AGGGTTATTGTCTCATGAGCGGATACATATTTGAATGTATTTAGAAAAAT
AAACAAATAGGGGTTCCGCGCACATTTCCCCGAAAAGTGCCACCTGACGT
CTAAGAAACCATTATTATCATGACATTAACCTATAAAAATAGGCGTATCA
CGAGGCCCTTTCGTCTTCAAGAATTAATTCATGGCTGACTAATTTTTTTT
ATTTATGCAGAGGCCGAGGCCGCCTCGGCCTCTGAGCTATTCCAGAAGTA
GTGAGGAGGCTTTTTTGGAGG

FIG. 4: DNA sequence of pNAS-094
GCGCGCGTTGACATTGATTATTGACTAGTTATTAATAGTAATCAATTACG
GGGTCATTAGTTCATAGCCCATATATGGAGTTCCGCGTTACATAACTTAC
GGTAAATGGCCCGCCTGGCTGACCGCCCAACGACCCCCGCCCATTGACGT
CAATAATGACGTATGTTCCCATAGTAACGCCAATAGGGACTTTCCATTGA
CGTCAATGGGTGGACTATTTACGGTAAACTGCCCACTTGGCAGTACATCA
AGTGTATCATATGCCAAGTACGCCCCCTATTGACGTCAATGACGGTAAAT
GGCCCGCCTGGCATTATGCCCAGTACATGACCTTATGGGACTTTCCTACT
TGGCAGTACATCTACGTATTAGTCATCGCTATTACCATGGTGATGCGGTT
TTGGCAGTACATCAATGGGCGTGGATAGCGGTTTGACTCACGGGGATTTC
CAAGTCTCCACCCCATTGACGTCAATGGGAGTTTGTTTTGGCACCAAAAT
CAACGGGACTTTCCAAAATGTCGTAACAACTCCGCCCCATTGACGCAAAT
GGGCGGTAGGCGTGTACGGTGGGAGGTCTATATAAGCAGAGCTCTCTGGC
TAACTAGAGAACCCACTGCTTACTGGCTTATCGAAATTAATACGACTCAC
TATAGGGAGACCCAAGCTGATCCACCGGTCGCCACCATGGTGAGCAAGGG
CGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCC
CGTGCCCTGGCCCACCCTCGTGACCACCTTCGGCTACGGCCTGCAGTGCT
TCGCCCGCTACCCCGACCACATGAAGCAGCACGACTTCTTCAAGTCCGCC
ATGCCCGAAGGCTACGTCCAGGAGCGCACCATCTTCTTCAAGGACGACGG
CAACTACAAGACCCGCGCCGAGGTGAAGTTCGAGGGCGACACCCTGGTGA
ACCGCATCGAGCTGAAGGGCATCGACTTCAAGGAGGACGGCAACATCCTG
GGGCACAAGCTGGAGTACAACTACAACAGCCACAACGTCTATATCATGGC
CGACAAGCAGAAGAACGGCATCAAGGTGAACTTCAAGATCCGCCACAACA
TCGAGGACGGCAGCGTGCAGCTCGCCGACCACTACCAGCAGAACACCCCC
ATCGGCGACGGCCCCGTGCTGCTGCCCGACAACCACTACCTGAGCTACCA
GTCCGCCCTGAGCAAAGACCCCAACGAGAAGCGCGATCACATGGTCCTGC
TGGAGTTCGTGACCGCCGCCGGGATCACTCTCGGCATGGACGAGCTGTAC
AAGTAAAGCGGCCCCTCTGCAGGATAATTCGGCACGAGGCTGTGTTAGAG
GTGAACCATCTTAATTACTAGTTCTATTACCTAATTCAGCTTCCTTGTTT
GGTCTGCTGTGGATCTGCCTTATTGCATATGCCATGCATCAGATAATGGA
TGCATCAGATAATGGTGTTAGACAAAGCTTCATTGTGAACAACCTAATGC
ATTTTAGAGAAACAATCTCATCACATTTTTTCTAGCCTTTCCTACATTTA
AACTTGCTGTTGCCCAAATTATAATTTTTTAAATGTCTTTGGTGGGCTTC
TGTTAATTCACATGACTTGAGCTTATAGCTATGTCTACTGCACAGATTGG
GTAATGGAACACTAAACTTTTATACTTGAAAATGACAGCCTTAAATGCTC
ATATCAGTCACAAATCTAGGATGTACTGTCTTGTTGTATGTGAGCTTTGT
AGAGATTTTTAAAAATATAAGCATCACCTTCCCATTGAAGAGTGGAGAGA
GTCTACTGGATGACTGGCCAGGAACTTTCTCTCTGAATCGGACATTTGGA
TGTCTTCTTTCTTCCAAGAAATGGTGGTTCACATTAAAGTATCATGGCCT
TATGTATGCTCAAATGGAATCTTATGTAACTTTCTTATTTAATTTTGGTC
TGCTTATTTTTAGATAAAATTGAAAGGAATTGTATAAATCAATTAACATA
TTAGCTGAGTTGTCCAACACATGGTATAAACGAATTACAACAGTAAACTA
TTACACATTTCCAAAAAAAAAAAAAAAAAAGCGGCCGCAAGCTTGGTACC
TCTAGAGGATCCGAACAAAAACTCATCTCAGAAGAGGATCTGAATATGCA
TACCGGTCATCATCACCATCACCATTGAGTTTGATCCCCGGGAATTCAGA
CATGATAAGATACATTGATGAGTTTGGACAAACCACAACTAGAATGCAGT
GAAAAAAATGCTTTATTTGTGAAATTTGTGATGCTATTGCTTTATTTGTA
ACCATTATAAGCTGCAATAAACAAGTTGGGGTGGGCGAAGAACTCCAGCA
TGAGATCCCCGCGCTGGAGGATCATCCAGCCGGCGTCCCGGAAAACGATT
CCGAAGCCCAACCTTTCATAGAAGGCGGCGGTGGAATCGAAATCTCGTAG
CACGTGTCAGTCCTGCTCCTCGGCCACGAAGTGCACGCAGTTGCCGGCCG
GGTCGCGCAGGGCGAACTCCCGCCCCCACGGCTGCTCGCCGATCTCGGTC
ATGGCCGGCCCGGAGGCGTCCCGGAAGTTCGTGGACACGACCTCCGACCA
CTCGGCGTACAGCTCGTCCAGGCCGCGCACCCACACCCAGGCCAGGGTGT
TGTCCGGCACCACCTGGTCCTGGACCGCGCTGATGAACAGGGTCACGTCG
TCCCGGACCACACCGGCGAAGTCGTCCTCCACGAAGTCCCGGGAGAACCC
GAGCCGGTCGGTCCAGAACTCGACCGCTCCGGCGACGTCGCGCGCGGTGA
GCACCGGAACGGCACTGGTCAACTTGGCCATGGTTTAGTTCCTCAACTTG
TCGTATTATACTATGCCGATATACTATGCCGATGATTAATTGTCAACACG
TGCTGATCAGATCCGAAAATGGATATACAAGCTCCCGGGAGCTTTTTGCA
AAAGCCTAGGCCTCCAAAAAAGCCTCCTCACTACTTCTGGAATAGCTCAG
AGGCAGAGGCGGCCTCGGCCTCTGCATAAATAAAAAAAATTAGTCAGCCA
TGGGGCGGAGAATGGGCGGAACTGGGCGGAGTTAGGGGCGGGATGGGCGG
AGTTAGGGGCGGGACTATGGTTGCTGACTAATTGAGATGCATGCTTTGCA
TACTTCTGCCTGCTGGGGAGCCTGGGGACTTTCCACACCTGGTTGCTGAC
TAATTGAGATGCATGCTTTGCATACTTCTGCCTGCTGGGGAGCCTGGGGA
CTTTCCACACCCTCGATCGAGCTAGCTTCGTGAGGCTCCGGTGCCCGTCA
GTGGGCAGAGCGCACATCGCCCACAGTCCCCGAGAAGTTGGGGGGAGGGG
TCGGCAATTGAACCGGTGCCTAGAGAAGGTGGCGCGGGGTAAACTGGGAA
AGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTG
CCGCCAGAACACAGGTAAGTGCCGTGTGTGGTTCCCGCGGGCCTGGCCTC
TTTACGGGTTATGGCCCTTGCGTGCCTTGAATTACTTCCACCTGGCTCCA
GTACGTGATTCTTGATCCCGAGCTGGAGCCAGGGGCGGGCCTTGCGCTTT
AGGAGCCCCTTCGCCTCGTGCTTGAGTTGAGGCCTGGCCTGGGCGCTGGG
GCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTT
TTTCTGGCAAGATAGTCTTGTAAATGCGGGCCAGGATCTGCACACTGGTA
TTTCGGTTTTTGGGCCCGCGGCCGGCGACGGGGCCCGTGCGTCCCAGCGC
ACATGTTCGGCGAGGCGGGGCCTGCGAGCGCGGCCACCGAGAATCGGACG
GGGGTAGTCTCAAGCTGGCCGGCCTGCTCTGGTGCCTGGCCTCGCGCCGC
CGTGTATCGCCCCGCCCTGGGCGGCAAGGCTGGCCCGGTCGGCACCAGTT
GCGTGAGCGGAAAGATGGCCGCTTCCCGGCCCTGCTCCAGGGGGCTCAAA
ATGGAGGACGCGGCGCTCGGGAGAGCGGGCGGGTGAGTCACCCACACAAA
GGAAAAGGGCCTTTCCGTCCTCAGCCGTCGCTTCATGTGACTCCACGGAG
TACCGGGCGCCGTCCAGGCACCTCGATTAGTTCTGGAGCTTTTGGAGTAC
GTCGTCTTTAGGTTGGGGGGAGGGGTTTTATGCGATGGAGTTTCCCCACA
CTGAGTGGGTGGAGACTGAAGTTAGGCCAGCTTGGCACTTGATGTAATTC
TCGTTGGAATTTGCCCTTTTTGAGTTTGGATCTTGGTTCATTCTCAAGCC
TCAGACAGTGGTTCAAAGTTTTTTTCTTCCATTTCAGGTGTCGTGAACAC
GTGGTCGCGGCCGGGATCCACCGGTCGCCACCATGGTGAGCAAGGGCGAG
GAGCTGTTCACCGGGGTGGTGCCCATCCTGGTCGAGCTGGACGGCGACGT
AAACGGCCACAAGTTCAGCGTGTCCGGCGAGGGCGAGGGCGATGCCACCT
ACGGCAAGCTGACCCTGAAGTTCATCTGCACCACCGGCAAGCTGCCCGTG
CCCTGGCCCACCCTCGTGACCACCCTGACCTGGGGCGTGCAGTGCTTCAG
CCGCTACCCCGACCACATGAAGCAGCACGACTTCTTCAAGTCCGCCATGC
CCGAAGGCTACGTCCAGGAGCGCACCATCTTCTTCAAGGACGACGGCAAC
TACAAGACCCGCGCCGAGGTGAAGTTCGAGGGCGACACCCTGGTGAACCG
CATCGAGCTGAAGGGCATCGACTTCAAGGAGGACGGCAACATCCTGGGGC
ACAAGCTGGAGTACAACTACAACAGCCACAACGTCTATATCACCGCCGAC
AAGCAGAAGAACGGCATCAAGGCCAACTTCAAGATCCGCCACAACATCGA
GGACGGCAGCGTGCAGCTCGCCGACCACTACCAGCAGAACACCCCCATCG
GCGACGGCCCCGTGCTGCTGCCCGACAACCACTACCTGAGCACCCAGTCC
GCCCTGAGCAAAGACCCCAACGAGAAGCGCGATCACATGGTCCTGCTGGA
GTTCGTGACCGCCGCCGGGATCACTCTCGGCATGGACGAGCTGTACAAGT
AAAGCGGCCTCGAGAGATCTGGCCGGCTGGGCCCGTTTCGAAGGTAAGCC
TATCCCTAACCCTCTCCTCGGTCTCGATTCTACGCGTACCGGTCATCATC
ACCATCACCATTGAGTTTAAACCCGCTGATCAGCCTCGACTGTGCCTTCT
AGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGC
GGTGGGCTCTATGGCTTCTGAGGCGGAAAGAACCAGTGGCGGTAATACGG
TTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAG
AGGTGGCGAAACCCGACAGGACTATAAAGATACCAGGCGTTTCCCCCTGG
AAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATC
GTCTTGAGTCCAACCCGGTAAGACACGACTTATCGCCACTGGCAGCAGCC
ACTGGTAACAGGATTAGCAGAGCGAGGTATGTAGGCGGTGCTACAGAGTT
CTTGAAGTGGTGGCCTAAGTACGGCTACACTAGAAGGACAGTATTTGGTA
TCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCT
TTTCTACGGGGTCTGACGCTCAGTGGAACGAAAACTCACGTTAAGGGATT
TTGGTCATGACATTAACCTATAAAAATAGGCGTATCACGAGGCCCTTTCG
TCTCGCGCGTTTCGGTGATGACGGTGAAAACCTCTGACACATGCAGCTCC
CGGAGACGGTCACAGCTTGTCTGTAAGCGGATGCCGGGAGCAGACAAGCC
CGTCAGGGCGCGTCAGCGGGTGTTGGCGGGTGTCGGGGCTGGCTTAACTA
TGCGGCATCAGAGCAGATTGTACTGAGAGTGCACCATATATGCGGTGTGA
AATACCGCACAGATGCGTAAGGAGAAAATACCGCATCAGGCGCCATTCGC
CATTCAGGCTGCGCAACTGTTGGGAAGGGCGATCGGTGCGGGCCTCTTCG
CTATTACGCCA

1. A reporter construct comprising a reporter element and a target nucleic acid inserted 3′- to the reporter element into the untranslated region.

2. The reporter construct according to claim 1 wherein the reporter element is a gene or a cDNA or a functional fragment thereof.

3. The reporter construct according to claim 1 wherein the target nucleic acid is a gene, a cDNA, a DNA fragment or an expressed sequence tag.

4. The reporter construct according to claim 1 wherein the reporter gene codes for a light emitting protein, preferrably a fluorescent protein.

5. The reporter construct according to claim 1 wherein the reporter gene codes for yellow fluorescent protein, enhanced yellow fluorescent protein, green fluorescent protein or luciferase.

6. A method for the production of the reporter construct according to claim 1 comprising inserting a target nucleic acid 3′- to the reporter element into the untranslated region.

7. A method for the identification of biologically active oligo- or polynucleotides that modulate the expression of a target nucleic acid comprising using the reporter construct of claim 1.

8. A method for screening for the identification of biologically active oligo- or polynucleotides that modulate the expression of a target nucleic acid comprising transfecting a reporter construct according to claim 1 and a candidate oligo- or polynucleotide into a suitable cell line; and comparing the level of expression of the reporter protein when the reporter construct is transfected alone with the level of expression when the reporter construct and the oligo- or polynucleotide are transfected.

9. The method according to claim 8 wherein the biologically active oligo- or polynucleotides are antisense oligonucleotides.

10. The method according to claim 9 wherein the antisense oligonucleotides are phosphothioated antisense oligonucleotides or 2′-O-methoxy-ethyl antisense oligonucleotides.

11. The method according to claim 9 wherein the antisense oligonucleotides are chemically modified antisense oligonucleotides that allow RNAse H induction of mRNA cleavage.

12. The method according to claim 9 wherein the antisense oligonucleotides have a RNAse H independent biological effect on the expression of the reporter element.

13. Cells transfected or transformed with the reporter construct according to claim 1.
